(19) United States
(12) Patent Application Publication
(10) Pub. No.: US 2024/0000361 A1
(43) Pub. Date: Jan. 4, 2024

(54) ELECTRODE CATHETER

(71) Applicant: Japan Lifeline Co., Ltd., Tokyo (JP)

(72) Inventor: Takafumi WATANABE, Tokyo (JP)

(21) Appl. No.: 18/250,958

(22) PCT Filed: Dec. 27, 2021
(86) PCT No.: PCT/JP2021/048617

§ 371 Date: Apr. 27, 2023

(30) Foreign Application Priority Data
Mar. 10, 2021 (WO) PCT/JP2021/009686

(51) Int. Cl. A61B 5/283; A61B 5/00

(52) U.S. Cl. CPC A61B 5/283; A61B 5/6852; A61B 2562/0209; A61B 2562/222; A61B 2562/227

(57) ABSTRACT

An electrode catheter includes a catheter shaft, a connector connected to a proximal end side of the catheter shaft, at least one electrode mounted on a distal end side of the catheter shaft, and a lead wire connected to an inner peripheral surface of the at least one electrode at a distal end, extending through an interior of the catheter shaft, and connected to the connector at a proximal end. A ratio of an outer diameter of the lead wire to an outer diameter of the catheter shaft is from 0.12 to 0.35.

### CROSS REFERENCE TO RELATED APPLICATIONS

This application is a continuation of International Application No. PCT/JP2021/048617, filed on Dec. 27, 2021, which claims priority to International Application No. PCT/JP2021/009686, filed on Mar. 10, 2021, which is incorporated herein by reference.

### TECHNICAL FIELD

The disclosure relates to an electrode catheter.

### BACKGROUND

As an electrode catheter to be introduced into a coronary sinus or the like to measure an internal potential, an electrode catheter has been disclosed that includes a catheter shaft, a connector connected to a proximal end side of the catheter shaft, a coil spring connected to a distal end side of the catheter shaft, a plurality of ring electrodes mounted on a distal end portion of the catheter shaft, a distal end electrode mounted on a distal end of the coil spring, lead wires of the ring electrodes, a lead wire of the distal end electrode, and a core wire with a distal end portion connected to the distal end electrode and a proximal end portion connected to the connector (refer to JP 6780162 B).

### SUMMARY

For example, to smoothly introduce the catheter into a narrow blood vessel deep in the coronary sinus and measure the internal potential, it is desirable that the electrode catheter have an even smaller diameter (for example, an outer diameter of the shaft being 0.41 mm or less).

However, in an electrode catheter (coil spring and shaft) having such a small diameter, space for arranging the core wire and lead wires described above cannot be secured. As a result, an electrode catheter having a small diameter such as a shaft outer diameter of 0.41 mm or less has not actually been developed.

The disclosure has been made in view of the above-described circumstances. An object of the disclosure is to provide an electrode catheter typically having a small diameter for solving problems such as those described above.

An electrode catheter according to the disclosure includes a catheter shaft, a connector connected to a proximal end side of the catheter shaft, at least one electrode mounted on a distal end side of the catheter shaft, and a lead wire connected to an inner peripheral surface of the at least one electrode at a distal end, extending through an interior of the catheter shaft, and connected to the connector at a proximal end. A ratio of an outer diameter of the lead wire to an outer diameter of the catheter shaft is from 0.12 to 0.35.

According to the disclosure, it is possible to provide an electrode catheter having a diameter smaller than that in the related art.

### BRIEF DESCRIPTION OF DRAWINGS

FIG. 1 is a front view of an electrode catheter according to a first embodiment.

FIG. 2A is a partial cutaway front view of a main portion (portion IIA) of the electrode catheter illustrated in FIG. 1.

FIG. 2B is a partial cutaway front view of a main portion (portion IIB) of the electrode catheter illustrated in FIG. 1.

FIG. 2C is a partial cutaway front view of a main portion (portion IIC) of the electrode catheter illustrated in FIG. 1.

FIG. 2D is a partial cutaway front view of a main portion (portion IID) of the electrode catheter illustrated in FIG. 1.

FIG. 3A is a cross-sectional view taken along IIIA-IIIA in FIG. 2A.

FIG. 3B is a cross-sectional view taken along IIIB-IIIB in FIG. 2A.

FIG. 3C is a cross-sectional view taken along IIIC-IIIC in FIG. 2B.

FIG. 3D is a cross-sectional view taken along IIID-IIID in FIG. 2C.

FIG. 3E is a cross-sectional view taken along IIIE-IIIE in FIG. 2C.

FIG. 4A is a detailed view of portion IVA in FIG. 2A.

FIG. 4B is a detailed view of portion IVB in FIG. 2A.

FIG. 5 schematically illustrates a hardness gradient of a catheter shaft based on lead wires.

FIG. 6 schematically illustrates an electrode catheter according to a second embodiment.

FIG. 7 schematically illustrates an electrode catheter according to a third embodiment.

FIG. 8 schematically illustrates the electrode catheter according to the third embodiment.

### DESCRIPTION OF EMBODIMENTS

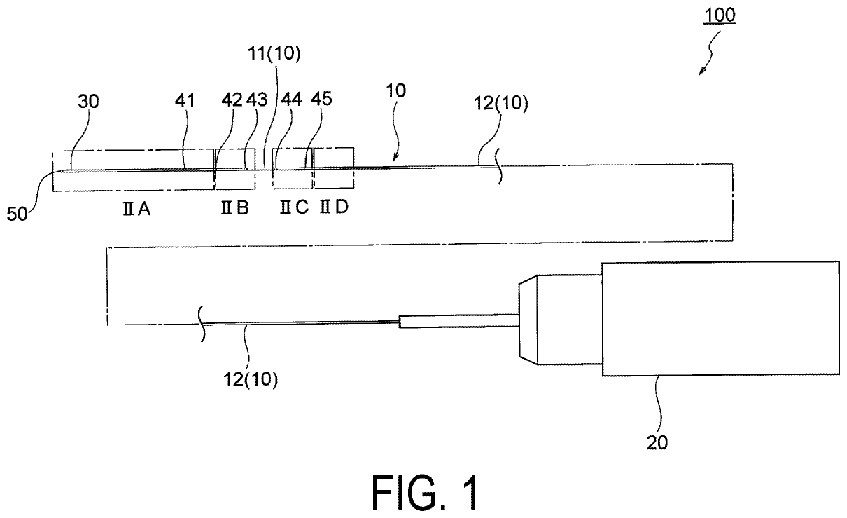

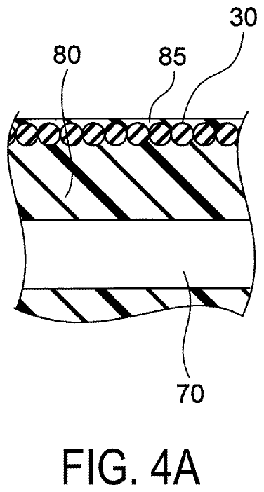

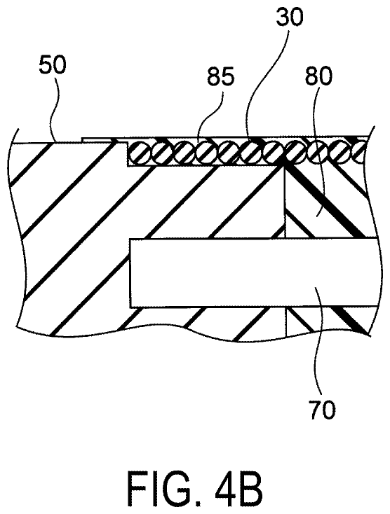

An electrode catheter 100 according to a first embodiment illustrated in FIGS. 1 to 4 (FIGS. 4A and 4B) is used, for example, to measure a potential at a site such as a pulmonary vein of a heart. The electrode catheter 100 includes a catheter shaft 10, a connector 20 connected to a proximal end side of the catheter shaft 10, a coil spring 30 connected to a distal end side of the catheter shaft 10, ring electrodes 41 to 45 mounted on a distal end portion or a distal end side of the catheter shaft 10, a distal end electrode 50 mounted on a distal end of the coil spring 30 provided at the distal end portion or the distal end side of the catheter shaft 10, lead wires 61 to 65 of the ring electrodes 41 to 45 respectively connected to inner peripheral surfaces of the ring electrodes 41 to 45 at distal ends, extending through an interior of the catheter shaft 10, and connected to the connector 20 at proximal ends, and a core wire 70, as a lead wire of the distal end electrode 50, connected to the distal end electrode 50 at a distal end, extending through the interiors of the coil spring 30 and the catheter shaft 10, and connected to the connector 20 at a proximal end. The core wire 70 is formed by coating a conductive wire made of a metal having an electrical conductivity of 1×10⁷ S/m or greater and a tensile strength of 500 N/mm² or greater with a resin. An outer diameter (D) of the catheter shaft 10 is from 0.30 to 0.41 mm, and a ratio (d1/D) of an outer diameter (d1) of the core wire 70 to the outer diameter (D) of the catheter shaft 10 is from 0.12 to 0.35, and preferably from 0.15 to 0.35.

The electrode catheter 100 includes the catheter shaft 10, the connector 20, the coil spring 30, the ring electrodes 41 to 45, the distal end electrode 50, the lead wires 61 to 65, and the core wire 70. The catheter shaft 10 includes a shaft distal end portion 11 and a shaft proximal end portion 12. A length (effective length) of the catheter shaft 10 is typically from 800 to 2000 mm, preferably from 1000 to 1600 mm, and 1500 mm as one preferred example.

The outer diameter (D) of the catheter shaft 10 is from 0.30 to 0.41 mm, preferably 0.33 to 0.37 mm, and 0.37 mm as one preferred example. When the outer diameter (D) is 0.41 mm or less, the catheter shaft 10 can be smoothly introduced into a narrow blood vessel, which was impossible or difficult with an electrode catheter in the related art. An inner diameter of the catheter shaft 10 is from 0.2 to 0.28 mm, preferably 0.22 to 0.24 mm, and 0.23 mm as one preferred example.

The shaft proximal end portion 12 of the catheter shaft 10 is formed of a metal tube (hypo tube) including a slit 125 having a spiral shape formed in a distal end portion thereof. The metal tube has a single lumen structure, and examples of the metal constituting the shaft proximal end portion 12 include stainless steel, NiTi, and β titanium. With the shaft proximal end portion 12 being constituted by a metal tube, excellent kink resistance, torque transmissibility, push-in characteristics, and the like can be exhibited even when the outer diameter of the shaft is small.

At the distal end portion where the slit 125 having a spiral shape is formed, rigidity of the metal tube is lowered to some extent to impart flexibility. As a result, the shaft proximal end portion 12 having both high rigidity (excellent kink resistance and push-in characteristics) inherent to a metal tube and flexibility at the distal end portion can be provided. Further, with a certain degree of flexibility imparted to the distal end portion of the shaft proximal end portion, a sudden change in rigidity at a boundary between the shaft proximal end portion 12 and the shaft distal end portion 11 can be alleviated.

A length of the shaft proximal end portion 12 is typically from 700 to 1950 mm, preferably from 1200 to 1500 mm, and 1450 mm as one preferred example. A length of the distal end portion of the metal tube in which the slit 125 having a spiral shape is formed is typically from 20 to 300 mm, preferably from 30 to 150 mm, and 30 mm as one preferred example.

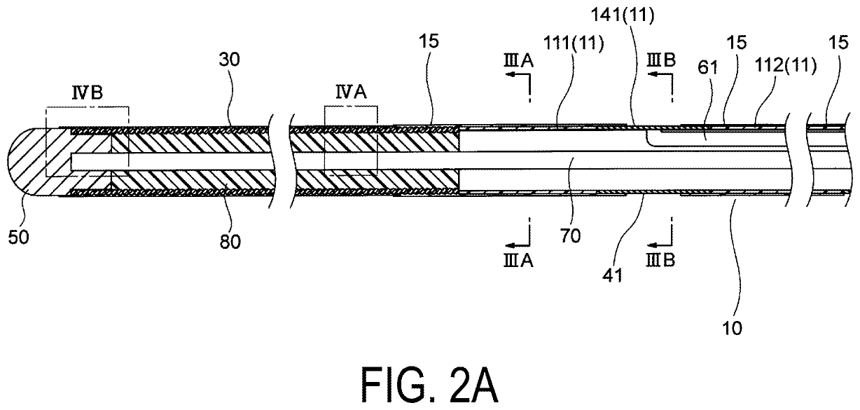

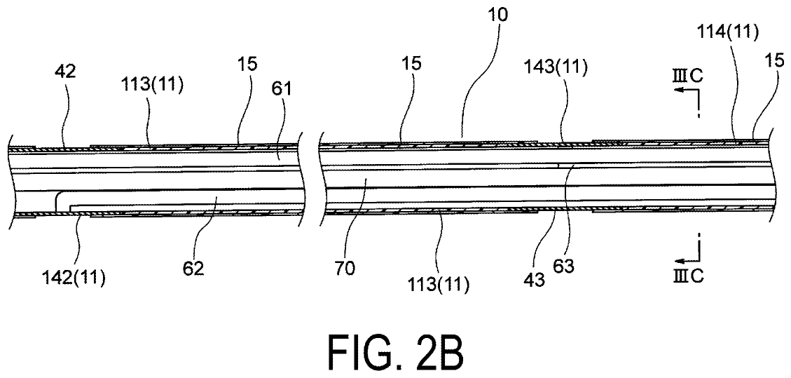

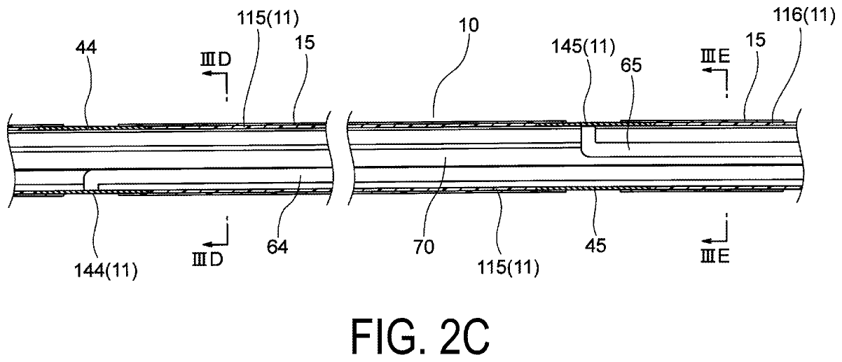

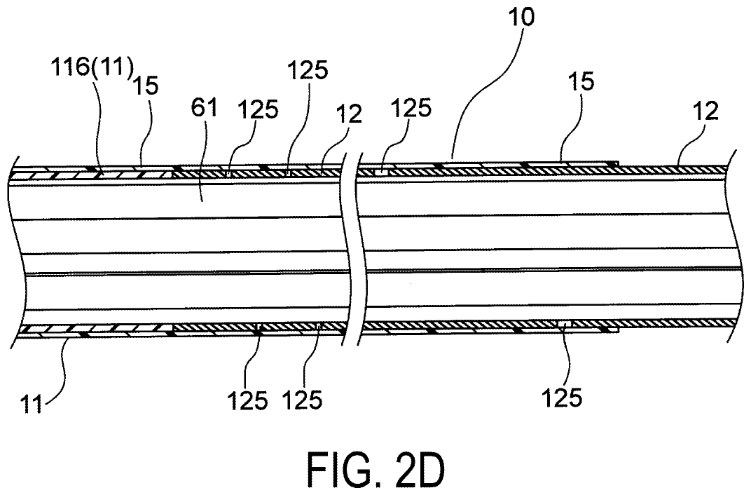

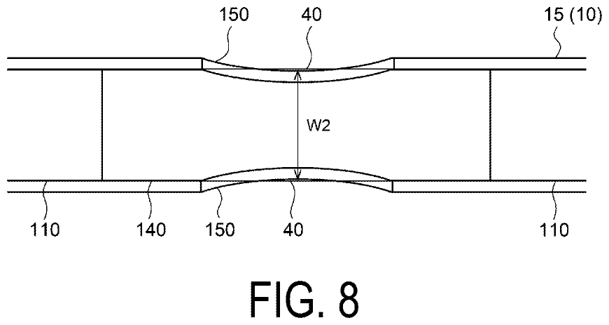

The shaft distal end portion 11 of the catheter shaft 10 includes a coupled structure formed by alternately coupling resin tubes 111 to 116 as non-metal tubes and metal rings 141 to 145 by bringing respective end surfaces thereof in contact with each other, and a resin coating layer 15 formed so as to coat an outer peripheral surface of the coupled structure. However, as illustrated in FIGS. 2A to 2C, at central portions of the metal rings 141 to 145 in a ring width direction (length direction of the catheter shaft 10), the resin coating layer 15 is peeled and removed across the entire outer peripheral surfaces, exposing the respective outer peripheral surfaces, and the ring electrodes 41 to 45 are constituted by these exposed outer peripheral surfaces. Note that, in the present embodiment, “ring electrode” refers to an electrode formed by exposing at least a portion of a surface or an outer peripheral surface of a “metal ring” having a ring shape. That is, the term “ring electrode” does not just refer to the surface of the “metal ring” being exposed in a ring shape. For example, as illustrated in FIGS. 7 and 8 described below, electrodes formed by exposing surfaces of the metal rings 141 to 145 in an elliptical shape or other non-ring shape are also the ring electrodes 41 to 45 in the present embodiment.

As illustrated in FIG. 2 (FIGS. 2A to 2D), in the coupled structure constituting the shaft distal end portion 11, the resin tube 111, the metal ring 141, the resin tube 112, the metal ring 142, the resin tube 113, the metal ring 143, the resin tube 114, the metal ring 144, the resin tube 115, the metal ring 145, and the resin tube 116 are coupled in this order from a distal end to a proximal end of the catheter shaft 10.

Examples of a constituent material of the resin tubes 111 to 116 include a polyether block amide copolymer resin (PEBAX (trade name)) and a urethane-based elastomer (Pellethane (trade name)). A length of the resin tubes 111 to 116 is typically from 1 to 5 mm, preferably from 2 to 4 mm, and 4 mm as one preferred example.

Examples of a constituent material of the metal rings 141 to 145 include metals having favorable thermal conductivity, such as aluminum, copper, stainless steel, gold, and platinum. To ensure favorable radiopacity for X-rays, however, platinum or the like is preferred. A width of the metal rings 141 to 145 is typically from 0.5 to 2 mm, preferably from 1 to 1.5 mm, and 1.2 mm as one preferred example. On the outer peripheral surfaces of both end portions of each of the metal rings 141 to 145, the resin coating layer 15 is layered over the entire periphery.

The resin coating layer 15 is formed by heat shrinking a heat-shrinkable tube, is fused to the resin tubes 111 to 116 constituting the coupled structure, and coats both end portions of each of the metal rings 141 to 145 over the entire periphery, thereby retaining the metal rings 141 to 145. The resin tubes 111 to 116 and the metal rings 141 to 145 have substantially the same outer diameters and inner diameters, and thus an outer diameter and an inner diameter of the shaft distal end portion 11 are substantially constant across the entire length thereof. As a result, the diameter of the shaft distal end portion 11 can be reduced and no step is formed on an inner peripheral surface of the shaft distal end portion 11. With this configuration, the lead wires 61 to 65 of the ring electrodes 41 to 45 and the core wire 70 can be smoothly inserted during manufacture of the electrode catheter 100.

A thickness of the resin coating layer 15 is typically from 0.01 to 0.055 mm, and preferably from 0.02 to 0.03 mm. A length of the shaft distal end portion 11 is typically from 20 to 55 mm, preferably from 24 to 30 mm, and 25 mm as one preferred example.

As illustrated in FIG. 2D, a proximal end surface of the coupled structure (resin tube 116) and a distal end surface of the shaft proximal end portion 12 (metal tube) are brought into contact with each other, and the resin coating layer 15 coating the outer peripheral surface of the coupled structure is formed up to a distal end portion of the metal tube in which the slit 125 is formed, thereby connecting the shaft distal end portion 11 and the shaft proximal end portion 12. Further, the coupled structure constituting the shaft distal end portion 11 and the metal tube constituting the shaft proximal end portion 12 have substantially the same outer diameter and inner diameter. As a result, the outer diameter and the inner diameter of the catheter shaft 10 are substantially constant across the entire length thereof.

With the proximal end surface of the coupled structure and the distal end surface of the shaft proximal end portion 12 being brought into contact with each other, and the point of contact and the outer peripheral surface of the distal end portion of the shaft proximal end portion 12 being coated with the resin coating layer 15, the shaft distal end portion 11 and the shaft proximal end portion 12 can be reliably connected to each other. Further, because the coupled structure constituting the shaft distal end portion 11 and the metal tube constituting the shaft proximal end portion 12 have substantially the same outer diameter and inner diameter, it is possible to prevent a step from being formed on an outer peripheral surface and an inner peripheral surface of a connecting portion between the shaft distal end portion 11 and the shaft proximal end portion 12.

With this configuration, since the diameter of the catheter shaft can be reduced and a step is not formed on the inner peripheral surface of the connecting portion between the shaft distal end portion 11 and the shaft proximal end portion 12, the lead wires 61 to 65 of the ring electrodes 41 to 45 and the core wire 70 can be smoothly inserted during manufacture of the electrode catheter 100. Furthermore, since the resin coating layer 15 is formed up to the distal end portion of the metal tube in which the slit 125 is formed, it is possible to prevent blood or the like from flowing into the interior of the catheter shaft 10 when the electrode catheter 100 is used.

The connector 20 is connected to the proximal end side of the catheter shaft 10 (shaft proximal end portion 12). The coil spring 30 is connected to the distal end side of the catheter shaft 10 (shaft distal end portion 11). A length of the coil spring 30 is typically from 5 to 25 mm, preferably 10 to 20 mm, and 15 mm as one preferred example.

An outer diameter of the coil spring 30 is from 0.25 to 0.35 mm, preferably from 0.28 to 0.33 mm, and 0.30 mm as one preferred example. An inner diameter of the coil spring 30 is from 0.15 to 0.29 mm, preferably from 0.18 to 0.25 mm, and 0.20 mm as one preferred example. Examples of a constituent material of the coil spring 30 include metal, platinum, tungsten, a platinum-tungsten alloy, stainless steel, and a nickel-titanium alloy.

As illustrated in FIGS. 2A, 4A, and 4B, the inside of the coil spring 30 is filled with a resin 80, and an insulating coating layer 85 made of the same resin as this filling resin is formed on an outer peripheral surface of the coil spring 30. With this configuration, integrity between the coil spring 30 and the core wire 70 is enhanced, and usability and the like can be improved. Further, with the insulating coating layer 85 formed on the outer peripheral surface of the coil spring 30, it is possible to maintain insulating properties of the coil spring 30 in the portion where the insulating coating layer 85 is formed.

As illustrated in FIG. 2A, a proximal end surface of the coil spring 30 and a distal end surface of the coupled structure (resin tube 111) are brought into contact with each other, and the resin coating layer 15 coating the outer peripheral surface of the coupled structure is formed up to a proximal end portion of the coil spring 30, thereby connecting the coil spring 30 and the catheter shaft 10.

The ring electrodes 41 to 45 are mounted on the catheter shaft 10 (shaft distal end portion 11). The ring electrodes 41 to 45 are formed by portions of the metal rings 141 to 145 constituting the coupled structure that are not coated with the resin coating layer 15 (portions peeled off and removed at the time of manufacture). A width (length in the axial direction) of the ring electrodes 41 to 45 is typically from 0.2 to 1.7 mm, preferably from 0.5 to 1 mm, and 0.5 mm as one preferred example.

The distal end electrode 50 is mounted on the distal end of the coil spring 30. The distal end electrode 50 is constituted by a distal end portion of a fixed portion (distal end rigid portion formed by solder) between the coil spring 30 and the core wire 70. As illustrated in FIG. 4B, although the insulating coating layer 85 is formed on the outer peripheral surface of the coil spring 30 at a rear end portion of the fixed portion between the coil spring 30 and the core wire 70, the insulating coating layer 85 is not formed (is peeled off and removed at the time of manufacture) at a distal end portion of the fixed portion, making it possible to configure the distal end electrode 50 with this distal end portion.

The core wire 70 is formed by coating a conductive wire made of a metal having a conductivity of 1×10⁷ S/m or greater and a tensile strength of 500 N/mm² or greater with a resin. The conductivity of the metal constituting the core wire 70 (conductive wire) is 1×10⁷ S/m or greater, and preferably 4.5×10⁷ S/m or greater. A core wire in which the conductivity of the constituent metal is less than 1×10⁷ S/m is not recommended for use as a lead wire of an electrode.

The tensile strength of the metal constituting the core wire 70 (conductive wire) is 500 N/mm² or greater, and preferably 1000 N/mm² or greater. A core wire in which the tensile strength of the constituent metal is less than 500 N/mm² does not have sufficient strength as a core wire required for an electrode catheter. Examples of metals satisfying the conductivity and the tensile strength described above include silver copper alloys, and preferable examples thereof include an Ag10Cu90 alloy (conductivity=4.5×10⁷ S/m, tensile strength=1000 N/mm²).

An outer diameter (d1) of the core wire 70 is typically from 0.065 to 0.1 mm, preferably from 0.07 to 0.09 mm, and 0.08 mm as one preferred example. A ratio (d1/D) of the outer diameter (d1) of the core wire 70 to the outer diameter (D) of the catheter shaft 10 is typically from 0.12 to 0.35, preferably from 0.15 to 0.35, more preferably from 0.18 to 0.28, and 0.22 (0.08 mm/0.37 mm) as one preferred example. Further, a ratio (d1²/D²) of a cross-sectional area of the core wire 70 to a cross-sectional area of the catheter shaft 10 is from 0.0144 to 0.1225, preferably from 0.0225 to 0.1225.

When the ratio (d1/D) is less than 0.15, sufficient strength cannot be imparted to the electrode catheter into which the core wire is inserted. Further, the core wire is more likely to come out from the distal end electrode 50 (fixed portion). On the other hand, when the ratio (d1/D) exceeds 0.35, sufficient space for inserting the core wire into the catheter shaft cannot be secured.

The lead wires 61 to 65 of the ring electrodes 41 to 45 are formed by coating a conductive wire made of the same metal as the constituent metal of the core wire 70 with a resin. An outer diameter (d2) of the lead wires 61 to 65 is preferably from 0.05 to 0.08 mm, and 0.065 mm as one preferred example.

A ratio (d2/D) of the outer diameter (d2) of the lead wires 61 to 65 to the outer diameter (D) of the catheter shaft 10 is from 0.12 to 0.35, preferably from 0.12 to 0.27, and 0.176 (0.065 mm/0.37 mm) as one preferred example. A ratio (d2²/D²) of a cross-sectional area of the lead wires 61 to 65 to the cross-sectional area of the catheter shaft 10 is from 0.0144 to 0.1225, and preferably from 0.0144 to 0.0729.

According to the electrode catheter 100 of the present embodiment, with the core wire 70 being formed by coating a conductive wire formed of a metal having a conductivity of 1×10⁷ S/m or greater and a tensile strength of 500 N/mm² or greater with a resin, and the value of the ratio (d1/D) described above being 0.15 or greater, the core wire 70 has both the physical properties (strength) required for a core wire and the conductivity required for a lead wire.

Accordingly, by disposing the core wire 70 in the interior of the coil spring 30 and the catheter shaft 10, it is not necessary to individually dispose a core wire and a lead wire as in the electrode catheter in the related art, and the outer diameters of the coil spring and the catheter shaft can be made sufficiently small, specifically, 0.41 mm or less.

Further, with the value of the ratio (d1/D) described above being 0.15 or greater, it is possible to prevent the core wire 70 from coming out of the distal end electrode 50. Further, with the value of the ratio (d1/D) being 0.35 or less, the core wire 70 can be inserted into the interior of the catheter shaft 10 together with the lead wires 61 to 65 with a margin.

Further, with the shaft distal end portion 11 being formed by the coupled structure, obtained by alternately coupling the resin tubes 111 to 116 and the metal rings 141 to 145 by bringing respective end surfaces into contact with each other, and the resin coating layer 15 coating the outer peripheral surface of the coupled structure (except for regions where the ring electrodes 41 to 45 are formed), it is possible to substantially prevent a step from being formed on the outer peripheral surface of the shaft distal end portion 11 and reliably reduce the diameter of the shaft distal end portion 11. Further, with the resin coating layer 15 being layered on both end portions of each of the metal rings 141 to 145, the ring electrodes 41 to 45 can be reliably mounted on the shaft distal end portion 11.

Further, with the proximal end surface of the coupled structure (resin tube 116) and the distal end surface of the shaft proximal end portion 12 (metal tube) being brought into contact with each other and the resin coating layer 15, coating the outer peripheral surface of the coupled structure, being formed up to the distal end portion of the shaft proximal end portion 12 (distal end portion of the metal tube including the region where the slit 125 is formed), the shaft distal end portion 11 and the shaft proximal end portion 12 can be reliably connected to each other. This also makes it possible to substantially prevent a step from being formed on the outer peripheral surface of the connecting portion between the shaft distal end portion 11 and the shaft proximal end portion 12.

Further, with the proximal end surface of the coil spring 30 and the distal end surface of the coupled structure (resin tube 111) being brought into contact with each other, and the resin coating layer 15, coating the outer peripheral surface of the coupled structure, being formed up to the proximal end portion of the coil spring 30, the coil spring 30 and the catheter shaft 10 can be reliably connected to each other.

Further, with the lead wires 61 to 65 of the ring electrodes 41 to 45 being formed by coating a conductive wire made of the same metal as that of the core wire 70 with a resin, and the ratio (d2/D) being 0.12 or greater, the lead wires 61 to 65 produce a reinforcing effect, similar to that of a core wire, on the catheter shaft 10. In particular, because the number of lead wires increases toward the proximal end side of the catheter shaft 10, the catheter shaft 10 increases in shaft strength toward the proximal end side, and an effect similar to that of a core wire is achieved with an increase in the outer diameter toward the proximal end side. Further, with the ratio (d2/D) being 0.27 or less, the lead wires 61 to 65 can be inserted into the interior of the catheter shaft 10 with a margin.

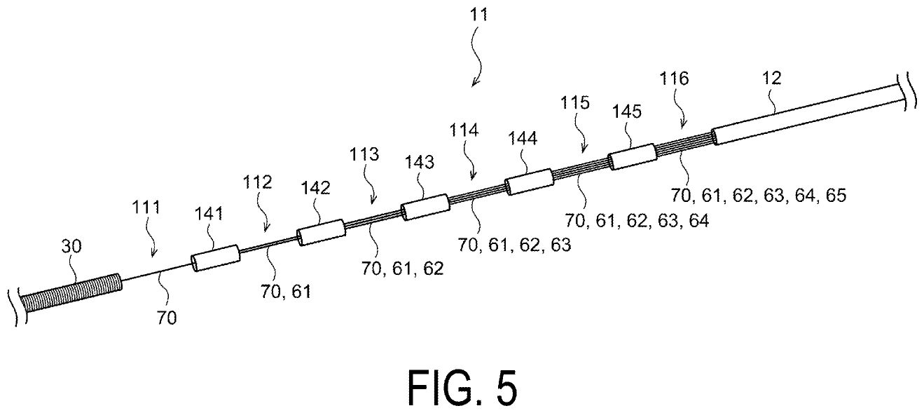

FIG. 5 schematically illustrates a hardness gradient or a strength gradient of the shaft distal end portion 11 based on the core wire 70 and the lead wires 61 to 65 described above. In FIG. 5, illustration of the resin tubes 111 to 116 and the resin coating layer 15 is omitted (only reference signs are provided for the resin tubes 111 to 116). As described above, the coil spring the first metal ring 141, the second metal ring 142, the third metal ring 143, the fourth metal ring 144, the fifth metal ring 145, and the metal tube 12 are disposed spaced apart from each other in the axial direction (direction connecting the proximal end and the distal end of the catheter shaft 10) by the first to sixth resin tubes 111 to 116, respectively, from the distal end (left end in FIG. 5) toward the proximal end (right end in FIG. 5) of the shaft distal end portion 11. Each interval between the coil spring 30 and each of the metal rings 141 to 145 (length of each of the resin tubes 111 to 116 in the axial direction) is, for example, 3 mm, and a length of each of the metal rings 141 to 145 in the axial direction is, for example, from 0.75 mm to 1.25 mm. In the coupled structure including the resin tubes 111 to 116 and the metal rings 141 to 145, a ratio of the length of each of the metal rings 141 to 145 in the axial direction to the length of each of the resin tubes 111 to 116 in the axial direction is preferably from 0.25 to 0.42.

In a first axial space between the core spring 30 and the first metal ring 141 in which the first resin tube 111 (not illustrated) is provided, the coil wire 70, serving as the lead wire of the distal end electrode 50 mounted on the distal end of the coil spring 30, extends in the axial direction. In a second axial space between the first metal ring 141 and the second metal ring 142 in which the second resin tube 112 (not illustrated) is provided, the first lead wire 61 of the first ring electrode 41 (first metal ring 141) extends in the axial direction together with the core wire extending from the first axial space to the proximal end side. In a third axial space between the second metal ring 142 and the third metal ring 143 in which the third resin tube 113 (not illustrated) is provided, the second lead wire 62 of the second ring electrode 42 (second metal ring 142) extends in the axial direction together with the core wire 70 and the lead wire 61 extending from the second axial space to the proximal end side.

In a fourth axial space between the third metal ring 143 and the fourth metal ring 144 in which the fourth resin tube 114 (not illustrated) is provided, the third lead wire 63 of the third ring electrode 43 (third metal ring 143) extends in the axial direction together with the core wire and the lead wires 61, 62 extending from the third axial space to the proximal end side. In a fifth axial space between the fourth metal ring 144 and the fifth metal ring 145 in which the fifth resin tube 115 (not illustrated) is provided, the fourth lead wire 64 of the fourth ring electrode 44 (fourth metal ring 144) extends in the axial direction together with the core wire 70 and the lead wires 61, 62, 63 extending from the fourth axial space to the proximal end side. In a sixth axial space between the fifth metal ring 145 and the metal tube 12 in which the sixth resin tube 116 (not illustrated) is provided, the fifth lead wire 65 of the fifth ring electrode 45 (fifth metal ring 145) extends in the axial direction together with the core wire 70 and the lead wires 61, 62, 63, 64 extending from the fifth axial space to the proximal end side.

As described above, a plurality of electrodes (distal end electrode 50 and first to fifth ring electrodes 41 to 45) are provided in the axial direction in the shaft distal end portion 11, and the number of lead wires (core wire 70 and first to fifth lead wires 61 to 65) increases toward the shaft proximal end portion 12. Therefore, as described above, the distal end side of the shaft distal end portion 11 can be flexibly bent, and a desirable hardness gradient for the catheter shaft in which the hardness increases toward the proximal end side can be naturally achieved without the use of a special member.

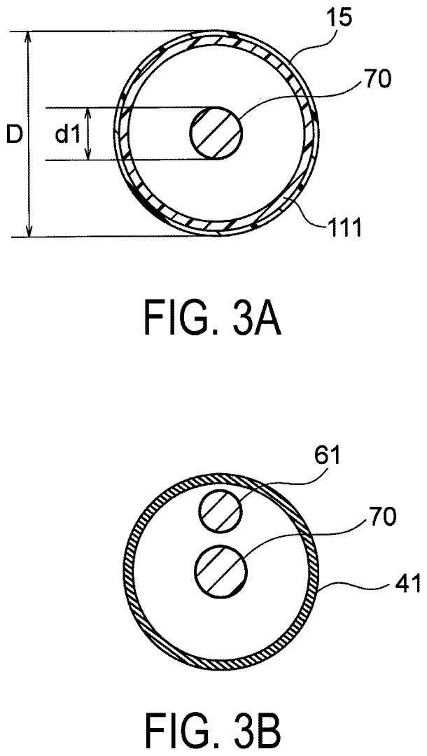

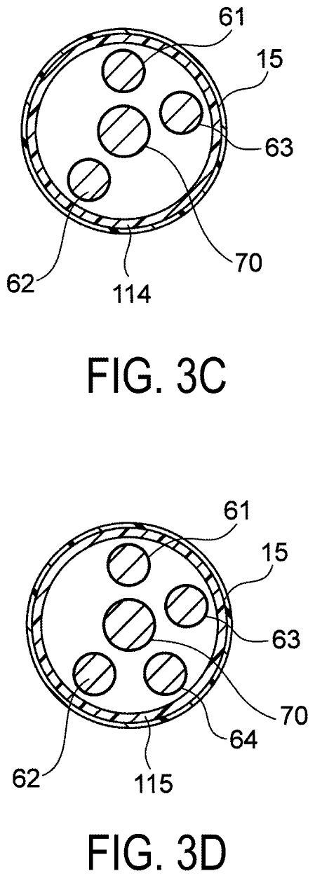

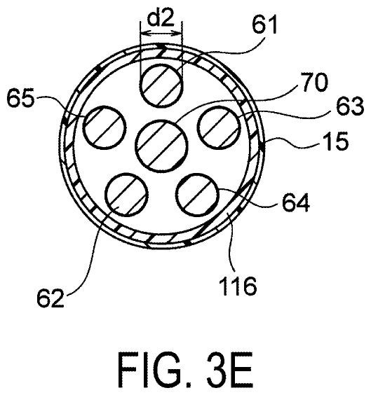

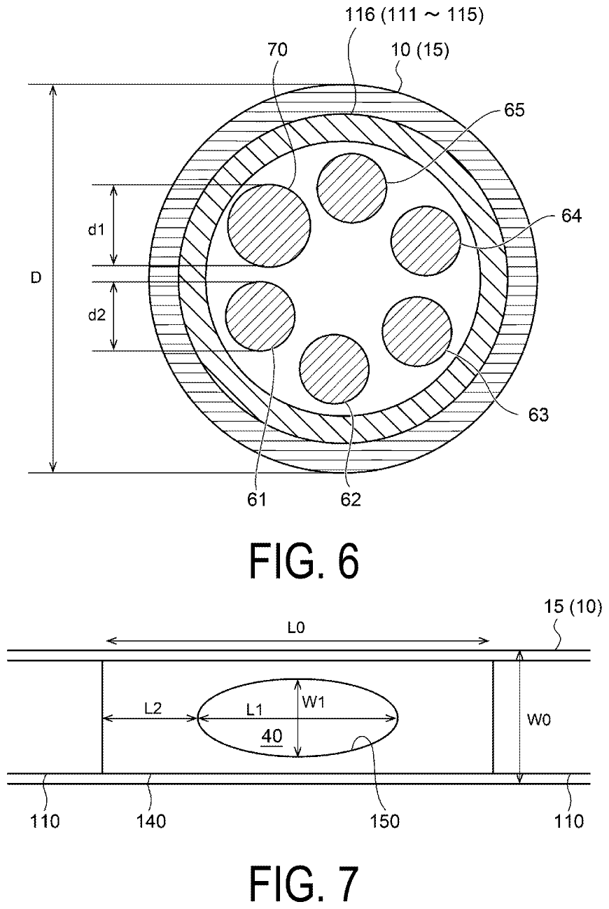

FIG. 6 schematically illustrates a second embodiment (corresponding to a modified example of FIG. 3E) for achieving a desired hardness gradient in the shaft distal end portion 11. FIG. 6 is a cross-sectional view of the sixth axial space between the fifth metal ring 145 and the metal tube 12 in FIG. 5, and also illustrates the sixth resin tube 116 and the resin coating layer 15 that are omitted in FIG. 5. An outer diameter of the resin coating layer 15 positioned at an outermost periphery of the catheter shaft 10 is the outer diameter D of the catheter shaft 10. While the first to fifth lead wires 61 to 65 are disposed around the core wire 70 in FIG. 3E, the core wire 70 in FIG. 6 is also disposed on substantially the same circumference as the first to fifth lead wires 61 to 65. However, since the sixth resin tube 116 is hollow, the core wire 70 and the first to fifth lead wires 61 to 65 can move about the positions illustrated in FIG. 6.

The outer diameter D of the catheter shaft 10 is, for example, from 0.3 to 0.6 mm, and the cross-sectional area of the catheter shaft 10 in this case is from 0.07 to 0.28 mm². The outer diameter d1 of the core wire 70 is, for example, 0.08 mm, and the cross-sectional area of the core wire 70 in this case is 0.005 mm². The outer diameter d2 of the first to fifth lead wires 61 to 65 is, for example, 0.065 mm, and the cross-sectional area of the first to fifth lead wires 61 to 65 in this case is 0.003 mm².

To achieve a desirable hardness gradient, the ratio (d1/D) of the outer diameter d1 of the core wire 70 to the outer diameter D of the catheter shaft 10 is preferably from 0.133 to 0.267, and the ratio of the cross-sectional area of the core wire 70 to the cross-sectional area of the catheter shaft 10 is preferably from 0.018 to 0.072. To achieve a desirable hardness gradient, the ratio (d2/D) of the outer diameter d2 of the first to fifth lead wires 61 to 65 to the outer diameter D of the catheter shaft 10 is preferably from 0.108 to 0.217, and the ratio of the cross-sectional area of the first to fifth lead wires 61 to 65 to the cross-sectional area of the catheter shaft 10 is preferably from 0.012 to 0.047. As a result of applying the preferable cross-sectional area ratios described above, a ratio of the total cross-sectional area of the lead wires 70, 61 to 65 as a whole to the cross-sectional area of the catheter shaft 10 in the sixth axial space (space in the sixth resin tube 116) where the core wire 70 and the first to fifth lead wires 61 to 65 are present is from 0.077 to 0.309.

Similarly, a ratio of the total cross-sectional area of the lead wires 70, 61 to 64 as a whole to the cross-sectional area of the catheter shaft 10 in the fifth axial space (space in the fifth resin tube 115) where the core wire 70 and the first to fourth lead wires 61 to 64 are present is from 0.065 to 0.261. A ratio of the total cross-sectional area of the lead wires 70, 61 to 63 as a whole to the cross-sectional area of the catheter shaft 10 in the fourth axial space (space in the fourth resin tube 114) where the core wire 70 and the first to third lead wires 61 to 63 are present is from 0.054 to 0.214. A ratio of the total cross-sectional area of the lead wires 70, 61 and 62 as a whole to the cross-sectional area of the catheter shaft 10 in the third axial space (space in the third resin tube 113) where the core wire 70 and the first and second lead wires 61, 62 are present is from 0.042 to 0.167. A ratio of the total cross-sectional area of the lead wires 70, 61 as a whole to the cross-sectional area of the catheter shaft 10 in the second axial space (space in the second resin tube 112) where the core wire 70 and the first lead wire 61 are present is from 0.030 to 0.119. A ratio of the total cross-sectional area of the lead wire 70 as a whole to the cross-sectional area of the catheter shaft 10 in the first axial space (space in the first resin tube 111) where the core wire 70 is present is from 0.018 to 0.072.

FIGS. 7 and 8 schematically illustrate a third embodiment in which the first to fifth metal rings 141 to 145 are exposed on the outer peripheral surface of the catheter shaft 10 as the first to fifth ring electrodes 41 to 45. FIG. 8 is obtained by rotating FIG. 7 by 90 degrees about a central axis of the catheter shaft 10. In these drawings, the first to sixth resin tubes 111 to 116 are representatively illustrated as a resin tube 110, the first to fifth metal rings 141 to 145 are representatively illustrated as a metal ring 140, and the first to fifth ring electrodes 41 to 45 are representatively illustrated as a ring electrode 40. As in the other embodiments described above, the coupled structure is formed by coating the resin tube 110 serving as a non-metal tube and the metal ring 140 for formation of the ring electrode 40 with the resin coating layer 15 from the outside, in a state in which end surfaces of the resin tube 110 and the metal ring 140 are in contact with each other.

The resin coating layer 15 includes an exposure window 150 that exposes at least a portion of the outer peripheral surface of the metal ring 140. The exposure window 150 is formed by coating the entire outer peripheral surface of the metal ring 140 (and the resin tube 110 and the like) with the resin coating layer 15 and then removing a portion of the resin coating layer 15 by a laser or the like. The outer peripheral surface of the metal ring 140 exposed to the outer peripheral surface of the catheter shaft 10 by the exposure window 150 is the ring electrode 40. As illustrated in FIG. 8, a plurality of (two in the example in FIG. 8) the exposure windows 150, that is, ring electrodes 40, are formed in the circumferential direction of the metal ring 140. Accordingly, regardless of the rotation angle in the body of the catheter shaft 10, the possibility that at least one of the plurality of ring electrodes 40 comes into contact with or comes close to body tissue or body fluid is increased, and thus measurement or treatment by the ring electrode 40 can be effectively performed. Further, as illustrated in FIG. 8, in a circumferential range of the metal ring 140 in which the exposure window 150 is not formed, the resin coating layer 15 is continuous in the axial direction (not interrupted by the exposure windows 150). Thus, strength of the resin coating layer 15 itself and the coupled structure coated with the resin coating layer 15 can be increased.

Each exposure window 150 has an axial length L1 greater than a width (arc length) W1 in the circumferential direction. As illustrated in FIG. 7, each exposure window 150 preferably has an elliptical shape, but may have another shape, such as a rectangular shape, a diamond shape, a circular shape, or a square shape. A length L0 of the metal ring 140 in the axial direction is, for example, from 0.75 mm to 1.25 mm, the length L1 of the major axis of the exposure window 150 in the axial direction is, for example, from 0.35 mm to 0.65 mm, and coated lengths L2 of the resin coating layer 15 on both sides of the major axis of the exposure window 150 are each, for example, from 0.2 mm to 0.3 mm. A ratio (L0/L1) of the length L1 of the exposure window 150 in the axial direction to the length L0 of the metal ring 140 in the axial direction is preferably from 0.35 to 0.65 to maintain high strength of the resin coating layer 15 and the coupled structure while enlarging the exposed area exposed by the exposure window 150 (that is, the area of each of the ring electrodes 40).

A diameter W0 of the catheter shaft 10 is, for example, from 0.3 mm to 0.6 mm, the width (arc length) W1 of the minor axis of the exposure window 150 in the circumferential direction is, for example, from 0.15 mm to 0.59 mm, and coated widths W2 on both sides of the minor axis of the exposure window 150 by the resin coating layer 15 are each, for example, from mm to 0.35 mm. A ratio (W1/πW0) of the width (W1) of the exposure window 150 in the circumferential direction to a circumferential length (πW0) of the catheter shaft 10 is preferably from 0.13 to 0.52 to maintain high strength of the resin coating layer 15 and the coupled structure while enlarging the exposed area exposed by the exposure windows 150 (that is, the area of each of the ring electrodes 40). In the example in FIG. 8 in which two exposure windows 150 are provided in the circumferential direction, a ratio (2W1/πW0) of a total width (2W1) of the two exposure windows 150 in the circumferential direction to the circumferential length (πW0) of the catheter shaft 10 is preferably greater than 0.26.

The present disclosure has been described above based on the embodiments. It should be understood by those skilled in the art that the embodiments are examples, that various modifications are possible in the combination of components and processing operations, and that such modifications are also within the scope of the present disclosure.

The present disclosure may be represented by the following items.
Item 1:
   An electrode catheter including a catheter shaft,
   a connector connected to a proximal end side of the catheter shaft,
   a coil spring connected to a distal end side of the catheter shaft,
   a ring electrode mounted on a distal end portion of the catheter shaft,
   a distal end electrode mounted on a distal end of the coil spring,
   a lead wire of the ring electrode connected to an inner peripheral surface of the ring electrode at a distal end, extending through an interior of the catheter shaft, and connected to the connector at a proximal end, and
   a core wire connected to the distal end electrode at a distal end, extending through the interior of the coil spring and the catheter shaft, and connected to the connector at a proximal end, wherein
   the core wire is formed by coating a conductive wire made of a metal having an electrical conductivity of 1×10⁷ S/m or greater and a tensile strength of 500 N/mm² or greater with a resin,
   an outer diameter (D) of the catheter shaft is from 0.30 to 0.41 mm, and
   a ratio (d1/D) of an outer diameter (d1) of the core wire to the outer diameter (D) of the catheter shaft is from 0.15 to 0.35.
Item 2:
   The electrode catheter according to Item 1, in which
   the catheter shaft is constituted by a shaft distal end portion having a coupled structure formed by alternately coupling a resin tube and a metal ring for ring electrode formation, the metal ring having substantially the same outer diameter as the resin tube, by bringing respective end surfaces into contact with each other, and a shaft proximal end portion formed of a metal tube.
Item 3:
   The electrode catheter according to Item 2, in which
   the shaft distal end portion is constituted by the coupled structure and a resin coating layer formed so as to coat an outer peripheral surface of the coupled structure,
   an outer peripheral surface of a central portion of the metal ring in a width direction is exposed without forming the resin coating layer across an entire periphery of the metal ring, and
   the ring electrode is constituted by the outer peripheral surface exposed.
Item 4:
   The electrode catheter according to Item 3, in which a proximal end surface of the coupled structure and a distal end surface of the shaft proximal end portion are brought into contact with each other, and this point of contact and an outer peripheral surface of at least the distal end portion of the shaft proximal end portion are coated with the resin coating layer, thereby connecting the shaft distal end portion and the shaft proximal end portion.
Item 5:
   The electrode catheter according to Item 4, in which a slit having a spiral shape is formed in at least a distal end portion of the shaft proximal end portion, and the outer peripheral surface of the shaft proximal end portion including a formation region of the slit is coated with the resin coating layer.
Item 6:
   The electrode catheter according to Item 4 or 5, in which a proximal end surface of the coil spring and a distal end surface of the coupled structure are brought into contact with each other, and this point of contact and an outer peripheral surface of a proximal end portion of the coil spring are coated with the resin coating layer, thereby connecting the coil spring and the catheter shaft.
Item 7:
   The electrode catheter according to any one of Items 1 to 6, in which an interior of the coil spring is filled with a resin, and an insulating coating layer made of the same resin as the resin for filling is formed on an outer peripheral surface of the coil spring.
Item 8:
   The electrode catheter according to Item 7, in which the distal end electrode is constituted by a distal end portion of a fixing portion configured to fix the core wire and the coil spring, the distal end portion being where the insulating coating layer is not formed.
Item 9:
   The electrode catheter according to any one of Items 1 to 8, in which
   the lead wire of the ring electrode is formed by coating, with a resin, a conductive wire made of a metal satisfying conditions required for a constituent metal of the core wire, and
   a ratio (d2/D) of an outer diameter (d2) of the lead wire to the outer diameter (D) of the catheter shaft is from 0.12 to 0.27.

### INDUSTRIAL APPLICABILITY

The disclosure relates to an electrode catheter.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. An electrode catheter comprising:
a catheter shaft;
a connector connected to a proximal end side of the catheter shaft;
at least one electrode mounted on a distal end side of the catheter shaft; and
a lead wire connected to an inner peripheral surface of the at least one electrode at a distal end, extending through an interior of the catheter shaft, and connected to the connector at a proximal end,
a ratio of an outer diameter of the lead wire to an outer diameter of the catheter shaft being from 0.12 to 0.35.

2. The electrode catheter according to claim 1, wherein
the at least one electrode includes a ring electrode mounted on the distal end side of the catheter shaft, and
a ratio of an outer diameter of the lead wire of the ring electrode to the outer diameter of the catheter shaft is from 0.12 to 0.27.

3. The electrode catheter according to claim 1, wherein the at least one electrode includes a distal end electrode mounted on a distal end of the electrode catheter, and
a ratio of an outer diameter of the lead wire of the distal end electrode to the outer diameter of the catheter shaft is from 0.15 to 0.35.

4. An electrode catheter comprising:
a catheter shaft;
a connector connected to a proximal end side of the catheter shaft;
at least one electrode mounted on a distal end side of the catheter shaft; and
a lead wire connected to an inner peripheral surface of the at least one electrode at a distal end, extending through an interior of the catheter shaft, and connected to the connector at a proximal end,
a ratio of a cross-sectional area of the lead wire to a cross-sectional area of the catheter shaft being from 0.012 to 0.072.

5. The electrode catheter according to claim 4, wherein
the at least one electrode includes a ring electrode mounted on the distal end side of the catheter shaft, and
a ratio of a cross-sectional area of the lead wire of the ring electrode to the cross-sectional area of the catheter shaft is from 0.012 to 0.047.

6. The electrode catheter according to claim 4, wherein the at least one electrode includes a distal end electrode mounted on a distal end of the electrode catheter, and
a ratio of a cross-sectional area of the lead wire of the distal end electrode to the cross-sectional area of the catheter shaft is from 0.018 to 0.072.

7. An electrode catheter comprising:
a catheter shaft;
a connector connected to a proximal end side of the catheter shaft;
an electrode mounted on a distal end side of the catheter shaft; and
a lead wire connected to an inner peripheral surface of the electrode at a distal end, extending through an interior of the catheter shaft, and connected to the connector at a proximal end,
the catheter shaft having a coupled structure formed by alternately coupling a non-metal tube and a metal ring for electrode formation by bringing respective end surfaces into contact each with each other.

8. The electrode catheter according to claim 7, wherein
at least a portion of an outer peripheral surface of the coupled structure is coated with a coating layer, and
the coating layer includes an exposure window configured to expose at least a portion of an outer peripheral surface of the metal ring.

9. The electrode catheter according to claim 8, wherein a length of the exposure window in an axial direction connecting a proximal end and a distal end of the catheter shaft is longer than a width of the metal ring in a circumferential direction.

10. The electrode catheter according to claim 9, wherein the exposure window has an elliptical shape.

11. The electrode catheter according to claim 8, wherein a ratio of the length of the exposure window in the axial direction to a length of the metal ring in the axial direction is from 0.35 to 0.65.

12. The electrode catheter according to claim 8, wherein a ratio of a width of the exposure window in a circumferential direction to a circumferential length of the catheter shaft is from 0.13 to 0.52.

13. The electrode catheter according to claim 8, wherein a plurality of the exposure windows are formed in the circumferential direction of the metal ring.

14. The electrode catheter according to claim 7, wherein
the catheter shaft includes a metal tube on a proximal end side of the coupled structure, and a slit having a spiral shape is formed in at least a distal end portion of the metal tube.

15. The electrode catheter according to claim 7, wherein a ratio of a length of the metal ring in an axial direction to a length of the non-metal tube in an axial direction is from 0.25 to 0.42.

16. The electrode catheter according to claim 1, wherein
a plurality of the at least one electrode are provided in an axial direction connecting a proximal end and a distal end of the catheter shaft, and
a number of the lead wires increases toward the proximal end side of the catheter shaft.
